(19) United States
(12) Patent Application Publication
(10) Pub. No.: US 2007/0276169 A1
(43) Pub. Date: Nov. 29, 2007

(54) METHODS FOR MONITORING HYDRATE INHIBITION INCLUDING AN EARLY WARNING SYSTEM FOR HYDRATE FORMATION

(75) Inventors: Bahman Tohidi, Midlothian (GB); Zahidah Zain, Wangsa Melawati (MY); Jinhai Yang, Edinburgh (GB); Rod Burgass, Linden (GB)

Correspondence Address: YOUNG & BASILE, P.C., 3001 WEST BIG BEAVER ROAD, SUITE 624, TROY, MI, 48084, US

(73) Assignee: Heriot-Watt University, Edinburgh (GB)

(21) Appl. No.: 11/719,140

(22) Filed: May 11, 2007

(30) Foreign Application Priority Data
Nov. 16, 2005 (GB) PCT/GB05/04420

(51) Int. Cl. C07C 9/00

(52) U.S. Cl. 585/15

(57) ABSTRACT

A method for determining the presence of a hydrate history in a fluid involves obtaining a sample of the fluid. The sample is then cooled to determine the amount of cooling required in order to induce hydrate formation. The result is then compared to the amount of cooling required to induce hydrate formation in a sample, which has been heat treated to remove any hydrate history present, before cooling. Also provided are methods for prevention or control of hydrate formation of a fluid in a system. The degree of inhibition of hydrate formation in the fluid is monitored and then the system conditions or fluid composition are adjusted as required. Methods for carrying out monitoring of a fluid liable to form hydrates are also described.

### RELATED APPLICATION

This application claims priority to PCT Application No. PCT/GB2005/04420 filed on Nov. 16, 2005.

### FIELD OF THE INVENTION

The present invention relates to the field of hydrate formation, the novel methods described herein are of particular applicability to the field of petroleum engineering, but are of interest to any industry where the monitoring of hydrate formation or a measure of water activity is important.

### BACKGROUND OF THE INVENTION

Gas hydrates are crystalline compounds friend as a result of a physical combination of water and suitably sized molecules, for example, C₁, C₂, C₃ hydrocarbons, or various combinations of the above. Preventing problems stemming from gas hydrate formation is a major flow assurance challenge in oil and gas production and transportation.

Known methods used to prevent problems stemming from hydrate formation include injecting thermodynamic inhibitors, for example methanol and glycol, or kinetic hydrate inhibitors (KHI's), for example the Hydtreat series of kinetic hydrate inhibitors developed and commercialised by Clariant Oil Services and discussed further at http://www.safewing.de/fun/internet.nsf/vwWebFramesets/8125EBEDDD8AE56EC1256D44003449FE?openDocument. The use of Kinetic inhibitors (KHI's) is also discussed in a report by Notz, et al. [Application of Kinetic Inhibitors to Gas Hydrate Problems, SPE Production & Facilities, November 1996], Argo, C. B., et al. [Commercial deployment of low-dosage hydrate inhibitors in a southern North Sea 69 km wet-gas sub-sea pipeline. SPE Production & Facilities 15 (2), 130-134, (2000)], and Phillips, N. S., and Grainger, M. [“Development and Application of Kinetic Hydrate Inhibitors in the North Sea”, SPE Gas Technology Symposium, Calgary, Canada, March 15-18, SPE 40030 (1998)].

Thermodynamic inhibitors act to prevent hydrate formation by reducing the activity of the water present in the system. Kinetic inhibitors act so as to delay hydrate formation.

Where thermodynamic inhibitors are used to prevent hydrate formation the current methods of determining the dosage required include determining the hydrate phase boundary for the fluid under consideration, experimentally and/or through modelling. The amount of thermodynamic inhibitor required to keep the fluid outside the hydrate stability zone, even when under the worst operating conditions, is then calculated and/or experimentally determined. Generally the worst conditions are those of the highest pressure and lowest temperature. Finally an estimated amount of inhibitor is added, including an allowance for what is lost from the aqueous phase to the vapour and liquid hydrocarbon phases, whilst also taking into account a safety margin.

The procedure for kinetic hydrate inhibitors (KHIs) is different. Calculation is based on the hydrate stability zone, (i.e. the conditions in which hydrate formation is thermodynamically favoured for a given fluid composition) and the worst operating conditions expected. A degree of subcooling, defined as the hydrate dissociation temperature minus the lowest expected operating temperature at the system pressure (T_{Equilibrium}−T_{Operating}),is considered. Then, fluid residence time in the pipeline is estimated. Following this, the inhibitor is experimentally tested at the same (or a slightly greater) degree of subcooling. The requirement of the inhibitor is that it prevents hydrate formation under these conditions for a period longer than the estimated fluid residence time in the system.

Practically, in most cases too much inhibitor is used. Excessive use of an inhibitor has environmental and economical consequences. However from an industrial standpoint this is viewed as acceptable, as the problems caused by the formation of hydrates have much greater economical consequences.

However, despite all these measures, hydrates do still form and cause serious operational and safety concerns. Their formation often takes place due to problems with injection pumps, a sudden increase in the water cut (water content of the fluid in the system), reducing the concentration of inhibitor, a change in fluid flow rate, emergency shut-downs, problems associated with start-ups, lack of reservoir fluid samples, lack of information on the composition of produced water, changes in the system temperature and pressure, uncertainty concerning the performance of the utilised inhibitor(s), and deleterious synergy between various inhibitors.

A further consideration in avoiding unplanned hydrate formation is related the phenomenon known as “hydrate history”. In a system that has previously contained hydrates or in which the nucleation of hydrates has already occurred, the degree or amount of cooling (at a given pressure), which is required to induce hydrate formation, can be substantially reduced. This can lead to hydrate formation occurring unexpectedly when a system is within a range of temperature and pressure conditions where hydrate formation would not normally be expected on the basis of the composition of the mixture present. This reduction in the amount of cooling or sub-cooling required to induce hydrate formation is attributed to residual structure of the water (“water memory”), or residual hydrate nuclei that can persist for prolonged periods in a system, where the fluid has not been heated above a temperature of the order of 30 to 35 deg C.

Experimental work has shown that preheating a sample will remove any hydrate history, as discussed by E. D. Sloan, S. Subramanian P. N. Matthews, J. P. Lederhos and A. A. Khokhar in Quantifying Hydrate Formation and Kinetic Inhibition [Ind. Eng. Chem.Res. 1998, 37, 3124-31321 where they report macroscopic evidence of residual structures using a sapphire multi-tube apparatus with a steel ball for a methane gas system with deionised water. They report that the apparent viscosity of the solution returned to baseline after heating to 28° C. and that residual liquid water was destroyed at this temperature. Makagon Y F. in Hydrates of hydrocarbon [Penwell Books Publishing Company, 1997 page 123-124 reports that in the heating of water above 30° C., the structured state of water almost completely disappears, and that the temperature at which the effect of initial hydrate formation is removed ranges from 30 to 35° C. He states that heating of water above these temperatures does not produce a noticeable difference.

It is an object of the invention to provide methods which avoid or minimise one or more of the foregoing disadvantages.

The novel methods described herein are based upon taking downstream measurements or on-line measurements in order to prevent hydrate formation and blockage.

### DESCRIPTION OF THE INVENTION

According to a first aspect the present invention provides a method for determining the presence of a hydrate history in a fluid comprising the steps of:

a) obtaining a sample comprising a fluid which may or may not have a hydrate history;

b) cooling the sample and determining the amount of cooling required to induce hydrate formation; and,

c) comparing the amount of cooling required to induce hydrate formation in the sample with that of, a heat treated sample which has been heat treated to remove any hydrate history present before cooling, in order to detect the presence or otherwise of a hydrate history in the said sample.

The heat treated sample may be the same sample as was first used to determine the amount of cooling required to induce hydrate formation. Alternatively it can be another similar sample of the fluid being investigated. Conveniently a single sample is taken and divided into two portions, one for cooling, and the other for heat treating followed by cooling. It is known that systems that have already or previously formed hydrates can form further hydrates without a high degree of cooling or “subcooling” due to the presence of a “hydrate history” as discussed above.

In the method according to one embodiment of the present invention, a sample of the fluid present within a system, which includes the water present (for example, the “produced water”, when considering a petroleum well product) is cooled and the degree of cooling necessary for hydrate formation is measured. Examples of methods for carrying out measurements to determine when hydrates are formed are described hereafter.

The results of this test are then compared with those for another similar sample, which has been preheated, typically to temperatures higher than 35° C., prior to cooling and hydrate formation. The second sample will determine the degree of cooling required for hydrate formation in the fluid where the possibility of there being a hydrate history present has been removed by the heating step. For a given system the amount of heating required to ensure removal of hydrate history, at the operating pressure, can be determined experimentally.

It is also possible to conduct the tests on the same sample by first cooling (using any of the various methods and systems described herein) the sample, to form hydrates and recording the degree of cooling required for hydrate formation and then heating the sample to the appropriate temperature, (typically to around 35° C.) to remove the hydrate history, prior to cooling again to form hydrates and recording the degree of cooling required.

Preferably the testing is carried out in an apparatus which has multiple sample cells which are rocked or otherwise agitated so that multiple measurements are undertaken under conditions which minimise the potential for stochastic deviation.

If the two tests, one for the unheated and one for the heated sample give close results the system fluid sample is unlikely to have previously experienced any hydrate formation.

However, if the first test shows less cooling is required for hydrate formation, there is a risk that hydrates are or have been present somewhere within the system; i.e. the measures taken to prevent hydrate formation are not effective and corrective action should be considered.

This method can therefore be implemented in the form of an early warning system enabling the user to react and prevent problems caused by hydrate formation.

The determination of hydrate formation can be carried out by any method, including but not limited to conductivity measurement, freezing point measurement, dielectric constant measurement, ultrasonic, pressure/temperature monitoring, or Fast Fourier Transform ultrasound, Quartz crystal microbalances can also be used to detect hydrate formation as a sample is cooled. The use of such methods is described hereafter in relation to other aspects of the invention.

This method of the invention can be carried out on samples obtained by any means. Advantageously on-line sampling is used and the samples are tested at the point they are taken. This provides the ability to monitor the fluid in the system and respond rapidly, to changes. For example, fluid flow in a pipeline can be diverted into a bypass loop for testing.

Sampling can also be carried out by means of pipeline “pigs”. The samples can be recovered from the pig and then tested according to the method. Alternatively the pig may contain suitable test equipment to carry out the method on a sample whilst in the pipeline and to report the results obtained. Pipeline pigs can also be used in the implementation of a method relating to another aspect of the invention.

According to a second aspect the present invention provides a method for investigating the potential of a fluid in a pipeline to form hydrates, the method comprising: providing a pipeline pig having a test cell containing a sample of a hydrate forming aqueous fluid composition; placing the pipeline pig in a pipeline to subject the sample in the test cell to the conditions of the pipeline; and recovering the pig from the pipeline and analyzing the sample contained in the test cell.

Pigs are regularly run in a pipeline. A conventional pig can be used when fitted with an appropriate test cell. Alternatively small “mini pigs” with can be used. It is possible to install a number of mini-pigs inside a carrier pig, which may be a conventional pipeline pig, and detect the hydrate formation potential within a pipeline. These mini-pigs are basically small test cells containing known composition aqueous phases. Various mechanisms could be used, for example, the small test cells could be equipped with one-way valves. If hydrates are formed the pressure inside the cell will drop, as some gas is used in hydrate formation, and more gas or other fluid will be introduced into the test cell from the pipeline. The mini-pigs may be deployed from the main pig. These mini-pigs could be recovered by the main pig, or left in the pipeline and recovered after a certain period of time by another pig, or they can be run on their own. Upon recovery, the pressure inside the cells can be measured, higher pressure than originally present would indicate the formation of hydrates had occurred in that cell. This information combined with the aqueous solution composition will give the hydrate forming characteristics of the pipeline. It is preferred to use seeding materials in the samples of the cells to minimise the degree of subcooling required for hydrate formation.

According to a third aspect the present invention provides a method for prevention or control of hydrate formation of a fluid in a system comprising the steps of: monitoring a degree of inhibition (i.e., safety margin) of hydrate formation in the fluid; and adjusting the system conditions or fluid composition.

A system may, for example, comprise subsea oil wells and their associated pipelines leading to petroleum production facilities. The adjustment to the system condition or fluid composition can be to avoid hydrate formation when hydrate formation is found or expected from the results of the monitoring. For, example more inhibitor can be injected into the fluid or alternatively the system pressure can be reduced. If on the other hand the degree of inhibition of hydrate formation is much higher than the design specifications then the inhibitor injection rate can be reduced. Alternatively the system conditions such as the pressure may be changed to avoid hydrate formation.

In order to monitor the system, any method may be employed to obtain samples, which can be analysed by methods described hereafter. For example, monitoring may be carried out by using pipeline pigs to obtain samples from a pipeline (flow line). The samples are then analysed in order to determine degree of inhibition of hydrate formation.

Preferably the monitoring is carried out in situ i.e. monitoring is by sampling and testing carried out at one or more location within the system. In situ monitoring can be carried out, for example, by means of a pipeline pig which is equipped with means for determining the degree of inhibition of hydrate formation. Alternatively a bypass to a flow line (a pipeline) of the system may be provided and equipped with monitoring means. Preferably the monitoring is continuous.

Preferably the response to the results of monitoring is automatic. For example, more or less hydrate inhibitor is injected into the system by command of an automated control system when the degree of hydrate inhibition falls outside pre-determined parameters.

Instead of the known practice of injecting inhibitors and assuming the amounts injected will be enough, the novel method disclosed herein involves monitoring the degree of inhibition at one or several points and/or to detect early hydrate formation, giving users enough time to react and prevent the formation of hydrates, for example by regulating the level of inhibitors within a system. Thus the method provides a technique for monitoring the hydrate safety margin and an early warning of hydrate formation. The monitoring can be carried out at any point in a system, for example in a pipeline downstream of an oil well production facility, or at or near the wellhead and/or at the receiving facilities. It also allows a user to observe the propensity of a system to form hydrates and/or wax (and other solids, such as asphaltene, salt and scale), enabling the user to reduce the amount of inhibitors within a system to an economical level. In addition to this, indications of hydrate formation at different points within a system can be gained, enabling the location and extent of inhibitor treatment to be ideally tailored to the particular situation.

The novel methods described herein can also be used to detect wax formation or the formation of any other material (e.g., asphaltene, scale and salt) due to changes in system temperature and/or pressure and/or fluid properties, using a similar procedure to that performed for the detection of hydrates, and any description of a method for the detection of hydrates and/or wax formation contained herein, unless explicitly limited otherwise, is to be read to include detection of all of such components.

A number of methods can be utilised for monitoring hydrate formation, and can be implemented as described hereafter with reference to preferred methods and examples.

### BRIEF DESCRIPTION OF THE DRAWINGS

Further preferred features and advantages of the present invention will appear from the following detailed description of some methods of implementation illustrated by means of experiments and illustrated with reference to the accompanying drawings in which:

FIG. 1(a) shows a graph of conductivity vs concentration measurements for poly(vinyl caprolactam) in water;

FIG. 1(b) shows a graph of conductivity for ethylene glycol/brine and methanol/brine systems;

FIG. 2 shows a graph of freezing point measurements;

FIG. 3 shows changes in resonance frequency of a quartz crystal microbalance when wax forms on it;

FIG. 4 shows a possible pipeline by-pass arrangement;

FIG. 5 shows a graph of dielectric constant variation for a sample, before and after heating;

FIG. 6 shows a graph of dielectric constant variation for a THF/water system; and,

FIG. 7 shows a graph of dielectric constant variation for natural gas hydrates.

### DESCRIPTION OF PREFERRED METHODS AND EXAMPLES

Conductivity Measurement

In systems a conductivity measurement taken on the fluid material within the system can provide a simple and cost effective method for monitoring the degree of hydrate inhibition within that system and give a warning against initial hydrate formation. This aspect of the present invention is of particular interest for systems containing produced water or organic inhibitors. It is known that the conductivity of a system is a function of the concentration of salts and/or organic inhibitors within that system. Therefore it is possible to relate the system's conductivity to the concentration of salts and/or organic inhibitors within that system. This can be used for monitoring the degree of hydrate inhibition (i.e., to calculate a “hydrate safety margin”), as detailed later.

On the other hand hydrates exclude salt and organic inhibitors from their structures as they form. Therefore, the formation of hydrates can result in significant and sudden changes in the system conductivity depending on the amount of hydrates formed (unlike what is generally expected as a result of water condensation or evaporation due to pressure and temperature changes in the system or pipeline). This could provide an early warning mechanism against massive hydrate formation and pipeline blockage. Therefore, a conductivity measurement at one or several points within the system or pipeline can provide an effective method for detecting initial hydrate formation, in particular in a controlled system (e.g., lab conditions).

However, salts and organic inhibitors affect water conductivity differently. An increase in salt concentration generally results in an increase in water conductivity, while an increase in organic inhibitor concentration generally may result in either a decrease or an increase in water conductivity, depending on the electrochemical composition of the hydrate inhibitor. However, assuming the composition of the produced water is generally known, it is possible to develop charts relating the conductivity of the combined salts and organic inhibitors to the concentration of organic inhibitor and/or presence of gas hydrates.

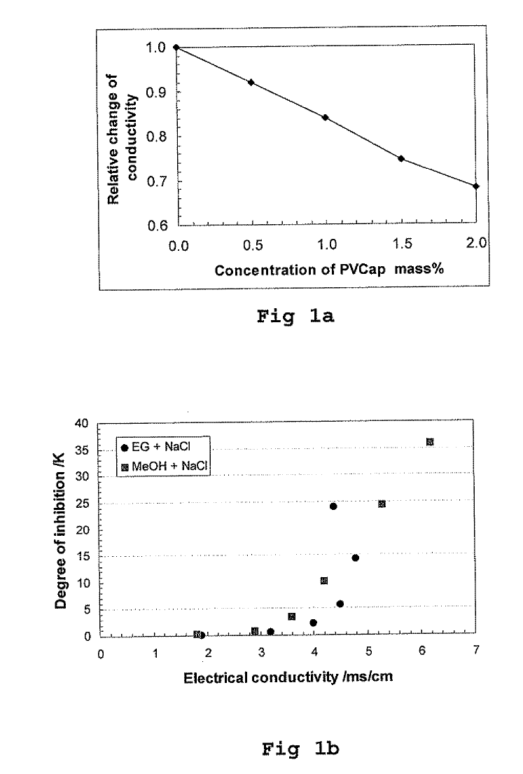

In practice, for example, the conductivity of the downstream water sample will be measured and this value will be used to estimate the concentration of organic inhibitor(s) and/or low dosage hydrate inhibitors present in the water phase using established charts generated for known produced water compositions. Then this information will be related to the degree of inhibition for the system. FIG. 1a shows the dependence of conductivity of an aqueous PVCap (poly [vinyl caprolactam]) solution on the PVCap concentration in water. The concentration of PVCap of a sample taken, for example from a pipeline, can therefore be determined with reference to the graph. FIG. 1b shows the relation between electrical conductivity and the degree of hydrate inhibition for an Ethylene glycol+brine system and for a Methanol+brine system, using 3 mass % NaCl in water, at 4° C. The conductivity of the aqueous phase can therefore be used for monitoring the concentration of the kinetic inhibitor in the system, and a hydrate safety margin based on the degree of hydrate inhibition, regarded as providing a safe operating condition, set.

Freezing point measurement

The freezing point depression of the aqueous phase within a system is directly related to the water activity within the system, hence the concentration of salts and/or organic inhibitors within the aqueous phase.

In order to determine the system's level of protection against hydrate formation, a sample of fluid liable to form hydrates including the aqueous phase is taken for freezing point measurement.

Various methods can be used in measuring the freezing point of aqueous solutions, including the method developed by Anderson, R., Llamedo, M., Tohidi, B., and Burgass, R. W. “Experimental Measurement of Methane and CO₂; Clathrate Hydrate Equilibria in Mesoporous Silica”, Journal of Physical Chemistry B, 107, 3507-3514, 2003].

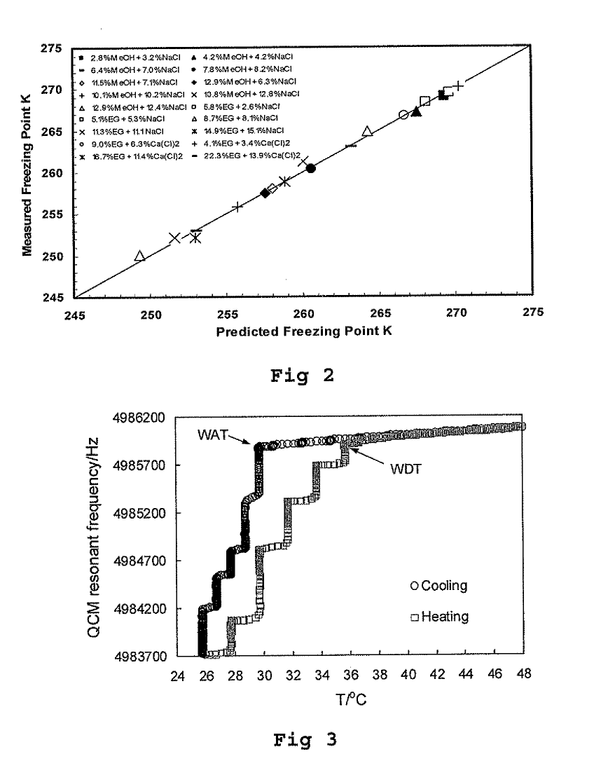

The measured depression in the freezing point can be directly related to the activity of water and therefore the hydrate inhibition characteristics of the aqueous solution. FIG. 2 shows the good correlation between actual and predicted freezing points for ethylene glycol/brine and methanol brine solutions.

It is also possible to undertake direct freezing point measurements by using a Peltier element and flow bypass as discussed later in this document.

Temperature and Pressure Measurement

Monitoring the temperature and/or pressure of a system, for example a pipeline system linking oil and/or gas wells and a production platform at selected locations can provide early warning information regarding hydrate formation. If a temperature measurement device is sufficiently sensitive and has a good response time, it can be used for detecting hydrate formation. Temperature measurement of the pipeline or its fluid content can be undertaken at selected points in the system. The local temperature will change when hydrates form due to the enthalpy of formation of the solid. Detection of the change can be used as an alert, which triggers a response such as injection of more inhibitor into the system. Similarly pressure measurement using appropriate sensors can also be used for detecting hydrate formation. For example, in a short section of pipe, a localised pressure drop can be expected as hydrate formation occurs. This can provide an early warning before complete blockage of the pipe occurs. Immediate remedial action, for example, reducing the system pressure to prevent further hydrate formation, can then be taken. (A pressure change generally travels much faster along a pipe than fluid flow. Therefore changing the system pressure may be preferred in such an emergency to injecting further inhibitors which may take some time to travel to the affected area of the pipe). Pressure monitoring, particularly at frequent intervals along a pipeline has other benefits. It can also be used to detect the presence of or movement of hydrate “plugs” that may be formed in a system. If a pipeline is blocked a sharp change in pressure over a short distance of the pipe can be expected. This allows a determination of the location of the plug, which can be important when considering the choice of method to be employed for removing the blockage.

Peltier Device with Quartz Crystal Microbalance

A Peltier Device is a known device made up of thermoelectric modules (TE) or thermoelectric coolers (TEC), as discussed by Pascal-Delannoy, F., Sorli, B. and Boyer, A. (2000).

A quartz crystal microbalance (QCM) is a known device, as discussed by Sauerbrey, G. [(1959). Verwendung von Schwingquartzen zur wagung dunner Schichten und zur Mikrowagung. Z. Phys., 155, 206-2221 and Loffelmann, M. and Mersmann, A. [(2002). How to measure supersaturation? Chemical Engineering Science, 57, 4301-4310].

Within the present invention as a whole it is preferable to use these devices for detection of hydrate (or wax) formation, although any other devices which perform the same or largely similar functions can be utilised. It is preferable that such devices operate with a rapid response and a high level of sensitivity over a wide working temperature and pressure range.

It is already known that a QCM can detect solid deposition, for example wax, hydrate, or scale, as discussed by Kraus, P. R., McClain, D. and Poindexter, M. K. in U.S. Pat. No. 5,734,098 (1998), Spates, J. J, Martin, S. J, and Mansure, A. J. in U.S. Pat. No. 5,661,233 (1996) and Burgass, R. W., Todd, A. C., Danesh, S. A. and Tohidi, K. B. in U.S. Pat. No. 6,298,724 (1998). A Quartz Crystal Microbalance (QCM) can be used as humidity sensor, as discussed in Sensors and Actuators, 84, (285-291).

A Peltier element or such like can provide various degrees of cooling/heating depending on the voltage/polarity applied to the system.

The detection of solid deposition on the surface of a QCM or such like gives an accurate indication of the level of hydrate formation within that system, as the resonant frequency (in Hertz) of the quartz crystal microbalance will drop considerably once solid particles/crystals of hydrates, wax, asphaltene and/or scale deposit on the surface of the QCM element.

Therefore, combining a QCM or similar with a Peltier Device or similar allows for the cooling/heating of a system and the consequent detection of solid deposition/removal within that system.

Preferably this should be undertaken by installing several QCM sensors or other detectors where different levels of subcooling, exist within a system, generally a pipeline. Preferably subcooling is induced to a required amount through the use of Peltier devices. These differing areas with different levels of subcooling can be used to determine the degree of inhibition against hydrate formation, as detailed below. They are referred to within this section of the description as ‘cells’.

For example one such area could be 1° C. colder than the temperature of the system or pipeline, with the others at decreasing increments of 1° C. (i.e. −1° C., −2° C., −3° C., . . . −10° C. of the system or pipeline temperature). Any increment may be used depending on the degree of precision of the monitoring required.

If hydrates form on the 1° C. area the implication can be drawn that the system is close to forming hydrates and more inhibitor should be used, whereas, if hydrates only form at high degrees of subcooling, for example at −10° C., the implication can be drawn that too much inhibitor is present (depending on the agreed safety margins with regard to the system, which are generally decided upon by the operators or owners of the system).

The novel method disclosed above can also be used to detect wax formation, using a similar procedure to that performed for the detection of hydrates.

For example, the application of a QCM for detecting Wax Appearance Temperature (WAT) and Wax Disappearance Temperature (WDT) in a stabilised North Sea condensate is demonstrated in FIG. 3. As shown in the figure, the formation of wax will cause a considerable reduction in the QCM resonant frequency, hence detecting wax formation. Seeding materials might be utilised in some applications to minimise the degree of subcooling required for wax or hydrate formation.

Isolating a System and Using a Peltier Element for Cooling and Heating

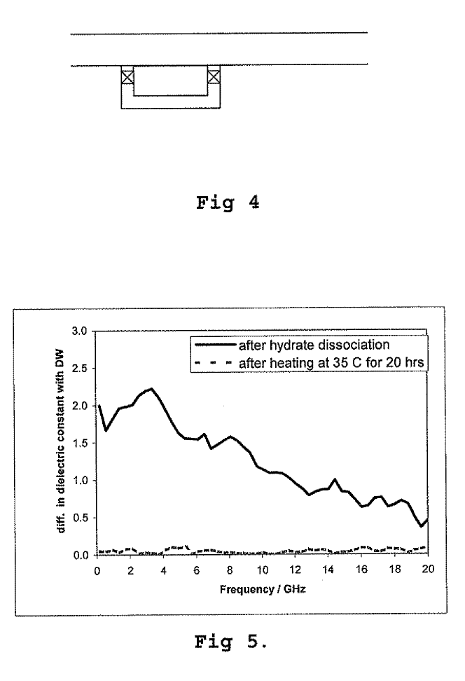

The present invention includes an arrangement whereby a bypass to the main system or pipeline is ‘hot-tapped’, preferably with two isolating valves. Preferably a Peltier element or such like is used for cooling and heating the bypassed section in order to detect hydrate formation, or the freezing point, or wax formation. Hydrate dissociation point can be determined by plotting pressure vs temperature and/or using a QCM or such like as in the above described arrangements. FIG. 4 illustrates schematically a main pipeline 1 bypassed with a pipeline loop 2.

As shown in FIG. 4 a bypass (pipeline loop 2) to the main flow line 1 is isolated by two isolating valves 4,5. This section of pipe is then used as a test cell. The bypassed section is preferably equipped with pressure and temperature measurement sensors with or without QCMs. Preferably, Peltier elements are used for cooling and heating the isolated section (hereinafter called ‘cell’). The cell is first cooled and then hydrate and/or wax formation is detected by plotting pressure vs temperature plot and/or utilising a QCM. The temperature for hydrate or wax formation is detected and recorded. The cell is then heated using the Peltier element and the hydrate dissociation or wax disappearance conditions are recorded. This information can then be used for establishing the precise hydrate and/or wax stability zone(s) under realistic system or pipeline conditions, using numerical techniques such as the thermodynamic modelling of hydrate and wax or correlation based methods. (Peltier elements are useful to cool a specific spot i.e a small area. For a required “isolated section” with a larger dimension/volume, it may be suitable to use other cooling/heating method such as, for example cooling coils. In addition, the Peltier elements are made of fragile semiconductors which can not bear any strain, and require restrict installation, for example, the flatness <0.02 mm, and with limited power.)

Afterwards, the system should be heated-up to around 35° C. (or higher in the case of wax) to remove any potential hydrate or wax particles and history. The precise temperature required to remove precipitated solid particles and/or hydrate history depends on system pressure as well as temperature, typically 35 deg C., is sufficient. Higher temperatures can be used if indicated by experiment.

The tests conducted in this laboratory confirm the removal of hydrate history after heating a system to 35° C. FIG. 5 shows the difference between dielectric constant of the system under investigation with that of Deionised Water (DW). The system investigated was a natural gas/water system. Hydrate was allowed to form at 4 deg C. and an appropriate pressure. Samples were then removed from the experimental rig and dielectric constant measurements made, at atmospheric pressure, at both 4 deg C. and then after heating to 35 deg C. As shown in the figure, there are significant differences between dielectric constants of the aqueous phase after hydrate dissociation and that of deionised water, indicating the presence of residual water structures (hydrate history). However, the difference between the dielectric constants of the aqueous phase and the deionised water is negligible after heating the system to 35° C. This proves that any remaining water structure (hydrate history), as a result of hydrate formation, will be totally removed after heating the system to 35° C. and higher.

Following this procedure the isolating valves are opened and the fluid in the cell is replaced with a new sample of the system or pipeline fluid and the test can be repeated at required time intervals.

Thus in this embodiment of the present invention, the bypassed section of the pipeline is converted into a small test cell with electronic connections for heating and cooling (directly or through a media) and data transfer.

Measuring the Dielectric Constant

Measurement of the dielectric constant of a material within a system enables the detection of the presence of small hydrate particles within that material, as the presence of small hydrate or ice particles results in a significant drop in the dielectric constant of that material.

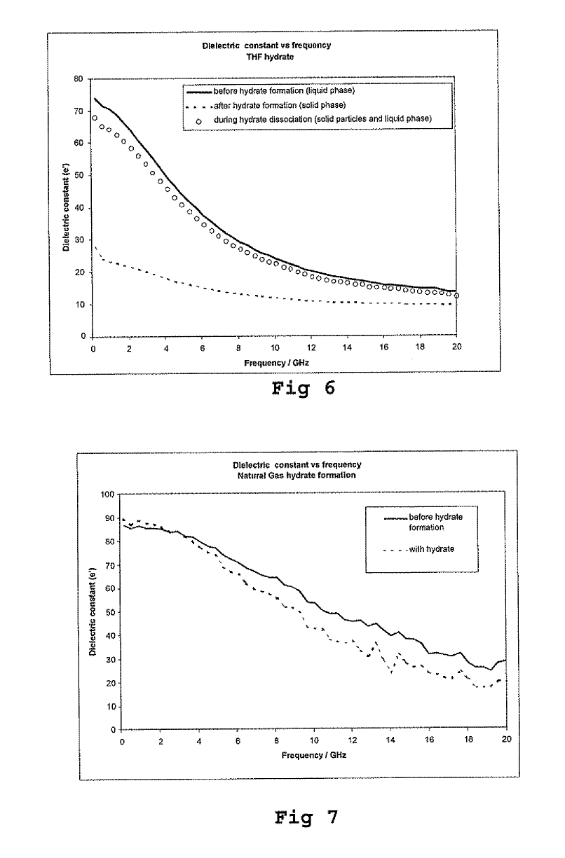

When hydrate formation is taking place, some free water molecules are converted into hydrates. These changes in structure of the water result in changes in its dielectric properties. As shown in FIG. 6, in the case of a water-soluble hydrate former such as tetrahydrofuran (THF, at 19 mass % in water), the value of dielectric constant is significantly reduced due to the presence of solid hydrates, as shown by the dotted line. In the presence of some hydrate particles (the curve represented by circles in the figure) the value of dielectric constant is lower than the mixture before hydrate formation, as shown by the smooth line. This is due to the fact that in the presence of solid or particles, the dipoles of water molecules will not be free or sufficient to re-orientate in the applied electrical field. Hence this results in lower dielectric constant. Similar evidence has been observed with regard to natural gas hydrates, as illustrated in FIG. 7.

If all the hydrates are dissociated, remnants of changes in the water history due to hydrate formation will remain for some time, depending on the system conditions such as temperature and pressure. Therefore this method can also monitor/detect hydrate formation, even if there are no solid hydrate particles present in the system.

A network analyser with an open-ended coaxial probe is preferably used to measure the dielectric properties of samples at microwave frequencies by a capacitive model. The capacitive model is described by Berube D. and Ghannouchi F. M. in A Comparative Study of Four Open-Ended Coaxial Probe Models for Permittivity Measurements of Lossy Dielectric/Biological Materials at Microwave Frequencies [1996, IEEE Transactions on Microwave Theory and Techniques, Vol. 44, no. 10, October]. The network analyser measures the reflection coefficient and impedance of the samples and calculates the dielectric properties.

Using Ultrasonic for Detecting Hydrate Nuclei

The ultrasonic signature (meaning amplitude of ultrasonic waves and the results of a Fast Fourier Transform (FFT) analysis performed on each waveform, (presented either directly in voltage or in non-dimensional form after being normalized) of a system containing hydrate nuclei differs from that of a system without any hydrates.

Within this method the ultrasonic signature of a sample of a system's fluid is investigated. The apparatus for measuring the ultrasonic signature can be directly connected to the pipeline (or a section of it, similar to the bypassed section described above) or the sample could be transferred to a laboratory for testing under pipeline pressure and temperature conditions. Any arrangement which allows for the passing of ultrasonic waves through a sample of the material within a system may be utilised.

Where hydrate history is being investigated, as described above with respect to the first aspect of the invention, an identical sample may be heated to temperatures above 35° C. to remove its hydrate history and then cooled to the same or largely similar sampling conditions, and its ultrasonic signature investigated.

Again if the results are similar, it can be concluded that there is no hydrate history in the sampled fluid. Otherwise, there is a risk of hydrate formation in the system.

### Some Particular Means of Utilising Methods of the Current Invention

The methods described above can be divided into three categories, as follows:

1. Methods based on conducting tests on two identical samples and comparing the results

2. Methods based on changing the system conditions and forming (and/or dissociating hydrates)

3. Integrations of the above two techniques

Examples of implementations of these categories of invention are as follows:

1. A sample of the fluid in a pipeline is taken. Depending on the technique used to take this sample, this sample could be only the aqueous phase of the material within the pipelines or a mixture with hydrocarbon phase. This sample is divided into two parts. One part is heated to temperatures higher than 35° C. to remove any history of hydrate formation. The other part is examined by taking ultrasonic signature and/or dielectric constant measurements as described above. The preheated sample is cooled to the test conditions and its ultrasonic signature and/or dielectric constants are measured and compared to the other sample's results in order to determine the sample's propensity to form hydrates and/or wax.

2. A sample of the fluid in a pipeline is isolated and the system is cooled to form hydrates and/or wax. In one embodiment a series of Peltier elements (with or without a fluid media) in combination with QCMs are used to establish various successive degrees of subcooling. The hydrate or wax formation is detected by monitoring the change in the QCM resonant frequency, giving an indication of the temperature conditions under which hydrates or wax tend to form, thus indicating the propensity of the system to form hydrates. In another embodiment, the Peltier element is used as a cooling and heating device for an isolated bypass section of the main pipeline. Hydrate or wax formation is detected by plotting pressure vs temperature readings, or frequency vs temperature in the case where a QCM is used. The system is then heated slowly to dissociate all hydrates or wax, and the hydrate dissociation point or wax disappearance temperature is observed. The system is then heated to remove any hydrate or wax history, prior to opening the isolating valves. In this method a part of the pipeline is converted into a small test cell.

3. A third embodiment of the invention described herein can be achieved by integrating the above techniques in order to detect the presence of any hydrate history in produced water. In this case the flow sample is diverted into two test sections. In one section the system is cooled to form hydrates, while in the second section the system is initially heated (for example to 35° C.) to remove any hydrate history prior to cooling to form hydrates. The two induction times and subcoolings are compared to detect the presence of hydrate history in the produced water.

### Benefits of the Current Invention

One benefit of the invention disclosed herein is that it allows the operators of a petroleum production facility or pipeline transportation system to cope with changes in the amount of water in a system, coming, for example, from a reservoir and/or condensation from the vapour phase within a pipeline. Similarly, other changes such as, for example seasonal changes in seabed temperature and/or pipeline pressure can also be accommodated. The invention provides early warning of the potential for hydrate formation, which can then be remedied by taking appropriate action.

For example, the invention disclosed herein enables appropriate changes to the inhibitor injection rate to be made.

The invention disclosed herein enables a user to monitor the degree of hydrate inhibition within a system, indicating how far the system is away from hydrate formation conditions.

Economical advantages come from eliminating the risk of hydrate blockage, the associated removal costs, and delayed production. It is also possible to determine if too much inhibitor is being used, hence allowing for the inhibitor dosage to be reduced, reducing associated costs and environmental impact.

Further advantages come from being able to detect minute hydrate particles carried within a medium.

Perhaps more importantly, detecting any history of hydrate formation, as hydrates may have dissociated by the time they have reached the sampling point, for example, as a consequence of flow through risers, where a combination of a reduction in the system pressure and an increase in the fluid temperature results in hydrate dissociation, is also possible. The economical benefits in this are significant, as initial hydrate formation does not generally result in pipeline blockage, rather it is the build up of hydrates which results in pipeline blockage.

The invention disclosed herein can provide an early warning for operators to take corrective measures to prevent pipeline blockage, which causes enormous cost, disruption, and delayed production.

Parts of the invention disclosed herein can also be used for detecting wax formation and/or determining the wax phase boundary and/or the performance of wax inhibitors.

There are no other current methods which offer these solutions.

1. A method for determining the presence of a hydrate history in a fluid comprising the steps of:
a) obtaining a sample comprising a fluid which may or may not have a hydrate history;
b) cooling the sample and determining the amount of cooling required to induce hydrate formation; and,
c) comparing the amount of cooling required to induce hydrate formation in the sample with that of, a heat treated sample which has been heat treated to remove any hydrate history present before cooling, in order to detect the presence or otherwise of a hydrate history in the said sample.

2. A method according to claim 1 wherein the heating to remove hydrate history is to at least 35 deg C.

3. A method according to claim 1 wherein the determination of hydrate formation is carried out by one or more of the following methods: conductivity measurement, freezing point measurement, dielectric constant measurement, ultrasound, pressure or temperature measurement.

4. A method for determining the presence of a hydrate history in a fluid according to claim 1 wherein Fast Fourier Transform ultrasound is used to determine the formation of hydrate nuclei in samples of the fluid.

5. A method according to claim 1 wherein the determination of hydrate formation is carried out by use of temperature controlled quartz crystal microbalances.

6. A method for the prevention or control of hydrate, wax, asphaltene, scale or salt formation of a fluid in a system comprising the steps of: monitoring a degree of inhibition of hydrate, wax, asphaltene, scale or salt formation in the fluid; and adjusting the system conditions or fluid composition in order to control the degree of inhibition of hydrate, wax, asphaltene, scale or salt formation.

7. A method according to claim 6 wherein the adjustment is to increase or decrease the concentration of hydrate inhibitors in the fluid.

8. A method according to claim 6 wherein the monitoring step comprises determining the presence of a hydrate history in the fluid by a. obtaining a sample fluid which may have a hydrate history;
b. cooling the sample to determine the amount of cooling required to induce hydrate formation; and
c. comparing the amount of cooling required to induce hydrate formation in the sample with that of a sample which has been heat-treated to remove any hydrate history before cooling.

9. A method according to claim 6 wherein samples of fluid from the system are obtained for monitoring in a bypass, isolable from a flow line containing the fluid to be investigated.

10. A method according to claim 6 wherein the adjustment of the system conditions or fluid composition is carried out automatically in response to a monitoring result.

11. A method according to claim 6 wherein samples of fluid for monitoring are obtained from the system by means of a pig introduced into a pipeline of the system.

12. A method according to claim 6 wherein the monitoring is carried out by means of a pipeline pig which comprises means for determining the degree of inhibition of hydrate, wax, asphaltene, scale or salt formation in the fluid of the system.

13. A method according to claim 6 wherein the monitoring comprises the steps of: determining the variation in conductivity of an aqueous phase of the fluid that occurs on changing the concentration of hydrate inhibitors and/or when hydrates are formed; and, monitoring the conductivity of the aqueous phase to determine the concentration of hydrate inhibitors present and/or determine if hydrate formation is occurring.

14. A method according to claim 6 wherein the monitoring comprises the steps of: determining the freezing point behaviour of an aqueous phase of the fluid with varying concentration of salts and organic inhibitors; and, measuring the freezing point of an aqueous phase of fluid of the system.

15. A method according to claim 6 wherein the monitoring comprises the steps of: determining the freezing point behaviour of an aqueous phase of the fluid with varying concentration of salts and organic inhibitors; and, measuring the freezing point of an aqueous phase of fluid of the system, the fluid being heated or cooled by means of a Peltier effect device and freezing point determined by pressure vs temperature or by changes in resonance frequency of a quartz crystal microbalance.

16. A method according to claim 6 wherein the monitoring comprises the steps of: providing at least one quartz crystal microbalance with temperature adjusting means; adjusting the temperature of the microbalance in the presence of the fluid in the system or a sample of fluid from the system; and monitoring the resonant frequency of the microbalance to determine the temperature at which hydrate, wax, asphaltene, scale or salt formation occurs.

17. A method according to claim 6 wherein the monitoring comprises the steps of: measuring the dielectric constant of the fluid of the system or a sample of the fluid of the system and comparing the observed measurement with measurement when no hydrate, wax, asphaltene, scale or salt or hydrate history is present.

18. A method according to claim 6 wherein the monitoring comprises detecting the formation of hydrate, wax, asphaltene, scale or salt nuclei during cooling of the sample, by means of ultrasound.

19. A method for investigating the potential of a fluid in a pipeline to form hydrate, wax, asphaltene, scale or salt, the method comprising: providing a pipeline pig having a test cell comprising a sample of a hydrate, wax, asphaltene, scale or salt forming aqueous fluid composition; placing the pipeline pig in a pipeline to subject the sample in the test cell to the conditions of the pipeline; and recovering the pig from the pipeline and analyzing the sample contained in the test cell.

20. A pipeline pig comprising means for determining the degree of inhibition of hydrate, wax, asphaltene, scale or salt formation in a fluid.
